(19) United States
(12) Patent Application Publication
(10) Pub. No.: US 2008/0139800 A1
(43) Pub. Date: Jun. 12, 2008

(54) Process For the Concentration and/or Isolation of Nucleic Acid or Nucleic Acid-Containing Species

(75) Inventors: Arne Deggerdal, Asker (NO); Evy H Reitan, Oslo (NO); Vidar Skagestad, Haslum (NO); Tine Thorbjornsen, Oslo (NO)

Correspondence Address: SEED INTELLECTUAL PROPERTY LAW GROUP PLLC, 701 FIFTH AVE, SUITE 5400, SEATTLE, WA, 98104, US

(73) Assignee: QIAGEN GmbH, Hilden (DE)

(21) Appl. No.: 10/557,124

(22) PCT Filed: Jun. 3, 2004
(86) PCT No.: PCT/EP04/05998

§ 371 Date: May 3, 2007

Related U.S. Application Data
(60) Provisional application No. 60/476,271, filed on Jun. 4, 2003.

(51) Int. Cl. C07H 1/00

(52) U.S. Cl. 536/25.41

(57) ABSTRACT

The present invention relates to a process for the concentration and/or isolation of nucleic acids or nucleic acid-containing species from a nucleic acid-containing solution, and a kit therefor. In one embodiment, the invention relates to the concentration and/or isolation of DNA and RNA from nucleic acid-containing solutions.

### BACKGROUND OF THE INVENTION

1. Field of the Invention

The present invention relates to a process for the concentration and/or isolation of nucleic acids or nucleic acid-containing species from a nucleic acid-containing solution, and a kit therefor. In one embodiment, the invention relates to the concentration and/or isolation of DNA and/or RNA from nucleic acid-containing solutions.

2. Description of the Related Art

Procedures involving the isolation and/or concentration of nucleic acids such as DNA and RNA continue to play a crucial role in biotechnology. Early methods of isolating nucleic acids involve a series of extractions using organic solvents, followed by ethanol precipitation and dialysis of the nucleic acids. These methods are relatively laborious and often result in a low nucleic acid yield.

According to U.S. Pat. No. 5,523,231, use of an alcohol such as ethanol (EtOH) or isopropanol at a concentration of about 70% (v/v) causes nucleic acids to precipitate around magnetically attractable beads but not to specifically bind to the beads. The precipitate can be separated from the supernatant by isolation of the magnetic beads by application of a magnetic field.

Later methods have taken advantage of the fact that nucleic acids are bound to silica surfaces under chaotropic conditions, that is typically 2 M to 8 M of a chaotropic salt, e.g. guanidinium salts, alone (see, e.g., U.S. Pat. No. 5,234,809; U.S. Pat. No. 5,234,909; U.S. Pat. No. 6,027,945), or in combination with EtOH (WO 95/01359). This methodology is typically performed either with a solid phase in form of a filter comprising a silica surface (e.g. spin columns from QIAGEN GmbH, Hilden, Germany) or in form of beads comprising a silica surface, e.g. paramagnetic silica beads (e.g. U.S. Pat. No. 6,027,945; U.S. Pat. No. 5,945,525; U.S. Pat. No. 5,658,548), or ferrimagnetic silica beads (WO 04/003231).

Regardless of the specific solid phase and nucleic acid-binding conditions, the volume of the nucleic acid-containing sample plays a pivotal role. The volume of the aqueous suspension, in which the nucleic acids or the nucleic acid-containing species are contained, will inevitably dilute the added components necessary for the binding of the nucleic acids. Therefore, an increasing amount of such components is needed in order to overcome this dilution effect, and, thus, have an appropriate final concentration of these key components.

Typically, for a chaotropic salt alone, a final concentration of 2 M to 8 M is needed to achieve an appropriate nucleic acid binding to a nucleic acid binding solid phase. If the chaotropic salt is used in combination with an alcohol, e.g. EtOH, the alcohol has typically a final concentration of 30-60% (v/v) to achieve an appropriate binding of the nucleic acids to a nucleic acid binding solid phase.

For many applications in which isolation of nucleic acids or nucleic acid-containing species is important, the nucleic acid-containing sample is an aqueous solution or has been brought into solution with a suitable solvent, e.g. a suitable buffer. If the sample reaches a critical volume, the isolation of the nucleic acids or the nucleic acid-containing species is not easily achieved by use of typical chaotropic binding conditions due to the dilution of the key components as mentioned above. This is a long known problem in the art and, thus, there is a requirement to solve this problem.

For some nucleic acid containing species, e.g. bacteria, the challenge of avoiding high sample volumes can be easily circumvented by centrifugation, and subsequently discarding the supernatant prior to lysis and/or binding. Free nucleic acids or small nucleic acid-containing species contained in a high volume of an aqueous solution, e.g. viruses in plasma, can neither be easily precipitated by centrifugation, nor can they be easily isolated under chaotropic conditions in the presence or absence of an alcohol.

Several methods have been reported for the precipitation of nucleic acids or small nucleic acid-containing species by other means than centrifugation. For instance, particles with a surface coated with amine groups are known to bind nucleic acids or nucleic acid-containing species in an aqueous solution. These particles do, however, have the pivotal disadvantage in view of the further purification that the nucleic acids are tightly bound to those beads and have to be eluted with a very high concentrated salt solution.

### BRIEF SUMMARY OF THE INVENTION

The present invention relates to a technology that overcomes the disadvantages of the methods known from the state of the art in binding nucleic acids from nucleic acid-containing aqueous solutions of a relatively large volume. By using the method of the present invention, the nucleic acids contained in an aqueous solution can easily be isolated and/or concentrated independent of the sample volume.

In general, the method according to the present invention comprises the steps of:
(a) providing an aqueous solution containing nucleic acids,
(b) adding an aliquot of substance I to (a),
(c) adding an aliquot of substance II to (b),
(d) centrifuge the aqueous solution of (c) and discard the supernatant.

The unexpected and beneficial effect of the present invention is independent of the order of the addition of substance I and substance II, which means that step (b) and step (c) are interchangeable. A crucial factor is the separated addition of substance I and substance II, which means that substance I and substance II can be added at the same time to the aqueous solution containing nucleic acids of step (a) but substance I and substance II should not be mixed prior to addition to the aqueous solution containing nucleic acids of step (a). Therefore, steps (b) and (c) as indicated above can be performed in reverse order or, alternatively, substance I and substance II can be added separately but at the same time. Thus, the present invention may also comprise the steps of:
(a) providing an aqueous solution containing nucleic acids,
(b) adding an aliquot of substance II to (a),
(c) adding an aliquot of substance I to (b),
(d) centrifuge the aqueous solution of (c) and discard the supernatant.
   or
(a) providing an aqueous solution containing nucleic acids,
(b/c) adding an aliquot of substance I and an aliquot of substance II separated from each other but at the same time to (a),
(d) centrifuge the aqueous solution of (b/c) and discard the supernatant.

In the nucleic acid-containing solution, substance I, which is the precipitating agent, will start to precipitate instantly in the presence of substance II, which is inducing the precipitation. The nucleic acids are part of the final precipitate either as a physical encapsulation in the emerging precipitates or via a specific affinity of the nucleic acids for the emerging precipitates.

The precipitate obtained in step (d) may be subjected to further purification steps utilizing standard methods. Several different methods are known in the art to further purify the so isolated and/or concentrated nucleic acids and can easily be applied by a skilled person.

Therefore, the present invention provides a method to isolate and/or concentrate nucleic acids from an aqueous solution as part of a precipitate independent of the volume of the aqueous solution.

The present invention has a broad application spectrum in biochemistry. As mentioned above, it is not easy to isolate viruses from an aqueous solution neither by centrifugation, nor can they be easily isolated under chaotropic conditions in the presence or absence of an alcohol. In another embodiment, the present invention can be utilized for the concentration and/or isolation of viruses from an aqueous solution, either as intact virus particles or as virus nucleic acids after virus lysis.

### DETAILED DESCRIPTION OF THE INVENTION

The present invention provides a method to isolate and/or concentrate nucleic acids from an aqueous solution as part of a precipitate independent of the volume of the aqueous solution. The method according to the present invention comprises the steps of:
(a) providing an aqueous solution containing nucleic acids,
(b) adding an aliquot of substance I to (a),
(c) adding an aliquot of substance II to (b),
(d) centrifuge the aqueous solution of (c) and discard the supernatant.

The unexpected and beneficial effect of the present invention is independent of the order of the addition of substance I and substance II, which means that step (b) and step (c) are interchangeable. A crucial factor is the separated addition of substance I and substance II, which means that substance I and substance II can be added at the same time to the aqueous solution containing nucleic acids of step (a) but substance I and substance II should not be mixed prior to addition to the aqueous solution containing nucleic acids of step (a). Therefore, steps (b) and (c) as indicated above can be performed in reverse order or, alternatively, substance I and substance II can be added separately but at the same time. Thus, the present invention may also comprise the steps of:
(a) providing an aqueous solution containing nucleic acids,
(b) adding an aliquot of substance II to (a),
(c) adding an aliquot of substance I to (b),
(d) centrifuge the aqueous solution of (c) and discard the supernatant.
   or
(a) providing an aqueous solution containing nucleic acids,
(b/c) adding an aliquot of substance I and an aliquot of substance II separated from each other but at the same time to (a),
(d) centrifuge the aqueous solution of (b/c) and discard the supernatant.

In the present invention, substance I and substance II are chosen in that way, that they form a heterogeneous solution. This means that in the nucleic acid-containing solution substance I, which is the precipitating agent, will start to precipitate instantly in the presence of substance II, which is inducing the precipitation. The nucleic acids are part of the final precipitate obtained in step (d) either as a physical encapsulation in the emerging precipitates or via a specific affinity of the nucleic acids for the emerging precipitates.

In the present invention, substance I is chosen from the group of negatively charged ionic detergents or is a mixture of such negatively charged ionic detergents. The term ‘negatively charged ionic detergents’ refers to ionic detergents which are negatively charged when dissolved in an aqueous solution, e.g. water, and when in addition the pH of the aqueous solution is in a range suitable for the isolation and/or concentration of nucleic acids. Such detergents as well as suitable solvents and a suitable pH range are well known to a skilled person. Preferably, substance I is lithium dodecyl sulfate (LiDS), sodium dodecyl sulfate (SDS) or a mixture thereof.

Substance I is added in a manner that the final concentration of substance I after addition of an aliquot thereof to the nucleic acid-containing aqueous solution of step (a) as well as after addition of an aliquot of substance II to the nucleic acid-containing aqueous solution of step (a), is in a range of from 0.1% (w/v) to 10% (w/v), preferably of from 0.4% (w/v) to 5% (w/v), and more preferably of from 0.5% (w/v) to 1% (w/v).

In the present invention, substance II is a chaotropic salt or a mixture of different chaotropic salts. It is well known to a person skilled in the art which salts have a chaotropic character. Preferably, the chaotropic component is selected from urea, sodium iodide, potassium iodide, sodium permanganate, potassium permanganate, sodium perchlorate, potassium perchlorate, sodium chlorate, potassium chlorate, guanidinium hydrochloride, guanidinium isothiocyanate, guanidinium thiocyanate, hexamine cobalt chloride, tetramethyl ammonium chloride, alkyltrimethyl ammonium chloride, tetraethyl ammonium chloride, tetramethyl ammonium iodide, alkyltrimethyl ammonium iodide, tetraethyl ammonium iodide, or is a mixture thereof. In the present invention, alkyl represents a branched or unbranched hydrocarbon radical having 1 to 20 carbon atoms.

Substance II is added in a manner that the final concentration of substance II after addition of an aliquot thereof to the nucleic acid-containing aqueous solution of step (a) as well as after addition of an aliquot of substance I to the nucleic acid-containing aqueous solution of step (a), is in a range of from 0.1 M to 7 M, preferably of from 0.2 M to 2 M, and more preferably of from 0.25 M to 1 M. In a preferred embodiment, the invention has the additional advantage that high concentrations of chaotropic components are not necessary.

Preferably, substance I and/or substance II are added to the nucleic acid-containing solution as a solution of suitable concentration. Every suitable solvent, e.g. water or a buffer system, can be applied to solubilize substance I and substance II. Any other suitable solvent according to the present invention is obvious to a skilled person. Alternatively, substance I and/or substance II can be added as solids.

The centrifugation mentioned in step (d) can be performed more or less directly subsequent to the addition of both substance I and substance II to the nucleic acids-containing solution due to the instant precipitation occurring after combining substance I and substance II in the nucleic acid-containing aqueous solution. Therefore, a time consuming incubation step is advantageously not required in the method according to the present invention.

The method of the present invention can be performed at any suitable temperature. A suitable temperature for such a method is obvious to a person skilled in the art. The preferred temperature range for the present invention is room temperature (18° C. to 25° C.).

The term ‘nucleic acid’ according to the invention comprises any nucleic acid and nucleic acid analog. The nucleic acid may, therefore, be, e.g., DNA or RNA or a mixture thereof. The source of the nucleic acid may be any imaginable source. It may either be a natural source, e.g. from cells or tissue, or an artificial source, e.g. a PCR product or the like. According to the invention, the nucleic acid has to be in an aqueous solution. The aqueous solution may be any natural solution, e.g. blood or cerebro-spinal fluid, or the nucleic acids or nucleic acid-containing species have to be brought into solution by any suitable solvent, e.g. a suitable buffer solution or the like. Such suitable solvents are obvious to a skilled person. If the nucleic acid source are cells, e.g. in a cell suspension or whole blood, the addition of substance I may advantageously additionally be used to lyse the cells. In this case an additional sufficient incubation time is needed to allow the cells to lyse. The required conditions to lyse cells, i.e. incubation time, temperature, concentration of the detergent etc., are well known to a person skilled in the art and can easily be adapted to the method according to the invention.

The precipitate obtained in step (d) can easily be separated from the solution by centrifugation or by other suitable means known to a person skilled in the art. Optionally, the precipitate obtained in step (d) can be subjected to further purification steps utilizing standard methods. Several different methods are known in the art to further purify the so isolated and/or concentrated nucleic acids and can easily be applied by a skilled person. In one exemplary and non-limiting embodiment, step (d) is followed by a purification comprising the rough steps of:
(e) resuspending the precipitate obtained in step (d) in a buffer containing chaotropic salt(s) and an alcohol, e.g. ethanol, and, subsequently, adding magnetic silica beads,
(f) allowing nucleic acids to bind to the magnetic beads, removing the magnetic beads after an appropriate incubation time and discarding the supernatant,
(g) exposing the complex of magnetic beads and nucleic acids to one or more washing steps
(h) elution of the nucleic acids from the magnetic beads.

In another aspect, the present invention provides a kit for the concentration and/or isolation of nucleic acids. The kit comprises at least substance I and substance II to perform the method of the present invention. For instance, substance I and substance II may be part of the kit as, e.g., solids or as stock solutions or as ready-to-use solutions. In a further aspect, the kit comprises in addition a set of solutions and/or devices to further purify the nucleic acids contained in the precipitate obtained in step (d). This set of solutions and/or devices should allow a further purification of the precipitate according to one of the several different methods known in the art, e.g. the above mentioned method.

### EXAMPLES

The following non-limiting examples are provided for the purpose of illustration.

### Example 1

### Pre-Concentration

50 μl of an aqueous solution of 5% (w/v) LiDS (Sigma, Deisenhofen, Germany) were added to one tube containing 1 ml of a DNA solution (1 μg/ml) and to one tube containing 1 ml of a RNA solution (1 μg/ml), respectively. Subsequently, 1 ml of 3.5 M guanidinium thiocyanate was added per tube. Instantly, a precipitate started to form. After 2 min incubation, the solutions were subjected to centrifugation (10000 g, 3 min) and the supernatants were discarded.

### Further Purification:

Each precipitate was further purified using the QIAGEN MagAttract RNA Cell Mini M48 kit (QIAGEN, Hilden, Germany) according to the manufacturers instructions.

### Results:

The yield of nucleic acids was 0.3 μg of RNA and 0.4 μg of DNA as quantified by measuring the UV absorbance. The OD_{260/280} (═OD at 260 nm/OD at 280 nm) for the DNA elute was 1.95 and the OD_{260/280} for the RNA elute was 2.1. Both, RNA and DNA, were easily amplified subsequently to isolation (QIAGEN QuantiTect RT-PCR kit and QIAGEN QuantiTect PCR kit, respectively, both of QIAGEN GmbH, Hilden, Germany).

### Example 2

1×10⁷ HL60 cells were resuspended in 1 ml of an aqueous solution of 10% (w/v) LIDS (Solution A). After sufficient incubation time for lysis (3 minutes at room temperature), aliquots of Solution A were added to aliquots of 5.5 M aqueous GTC solution (Solution B) as indicated in table 1. Subsequently, the solution was centrifuged (3000 g, 3 min) and the supernatant was discarded. The precipitate was washed once with 500 μl of Solution B.

Thereafter, the precipitate was further purified as described in Example 1. The results are listed in table 2.

TABLE 1

| No. | Solution A (μl) | Solution B (μl) |
|---|---|---|
| 1 | 10 | 990 |
| 2 | 100 | 900 |
| 3 | 200 | 800 |
| 4 | 400 | 600 |
| 5 | 500 | 500 |
| 6 | 800 | 200 |
| 7 | 900 | 100 |

TABLE 2

| | No. of | C_{[LiDS]} | | | | |
|---|---|---|---|---|---|---|
| | cells | (% | C_{[GTC]} | Total yield | Yield per | |
| No. | (×10⁶) | (w/v)) | (M) | (μg) | 10⁶ cells (μg) | OD_{260/280} |
| 1 | 1 | 0.1 | 5.4 | 0.3 | 0.3 | 1.86 |
| 2 | 2 | 1 | 5 | 1.2 | 0.6 | 2.02 |
| 3 | 3 | 2 | 4.4 | 2.2 | 0.8 | 2 |
| 4 | 4 | 4 | 3.3 | 2.4 | 0.4 | 1.99 |
| 5 | 5 | 5 | 2.8 | 2.9 | 0.6 | 1.85 |
| 6 | 8 | 8 | 1.1 | 2.8 | 0.4 | 2.06 |
| 7 | 9 | 9 | 0.6 | 1.4 | 0.2 | 2 |

### Example 3

### Pre-Concentration and Further Purification

1×10⁶ HL60 cells were lysed in 1 ml of an aqueous solution of 2% (w/v) LiDS as described in Example 2. Subsequently, 1 ml of 1 M aqueous guanidinium hydrochloride solution was added. Thereafter, the solution was centrifuged and the precipitate was further purified as described in Example 1.

### Results:

3.2 μg of nucleic acids (DNA and RNA) were isolated (OD_{260/280}=2.04).

### Example 4

### Pre-Concentration and Further Purification

1×10⁶ HL60 cells were incubated in 1 ml of an aqueous solution of 2% (w/v) SDS at pH 12.5. To lyse the cells efficiently, the suspension was incubated for 5 minutes at 90° C. The following steps were performed at room temperature. Subsequently, 1 ml of an aqueous solution of 2 M guanidinium hydrochloride was added. Thereafter, the solution was centrifuged and the precipitate was further purified as described in Example 1.

### Results:

0.5 μg of nucleic acids (DNA and RNA) were isolated (OD_{260/280}=2.04).

### Example 5

### Pre-Concentration and Further Purification

1×10⁶ HL60 cells were lysed in 1 ml of an aqueous solution of 2% (w/v) LiDS as described in Example 4. Subsequently, 1 ml of 2 M aqueous guanidinium hydrochloride solution was added. Thereafter, the solution was centrifuged and the precipitate was further purified as described in Example 1.

### Results:

1.6 μg of nucleic acids (DNA and RNA) were isolated (OD_{260/280}=2.12).

### Example 6

### Pre-Concentration and Further Purification

1×10⁶ HL60 cells were lysed in 1 ml of an aqueous solution of 2% (w/v) LiDS as described in Example 4. Subsequently, 1 ml of 2 M aqueous sodium iodide solution was added. Thereafter, the solution was centrifuged and the precipitate was further purified as described in Example 1.

### Results:

0.2 μg of nucleic acids (DNA and RNA) were isolated (OD_{260/280}=1.85).

### Example 7

### Pre-Concentration and Further Purification

400 μl of an aqueous solution of 2% (w/v) LiDS were added to 100 μl of an over-night culture of E. coli (OD=0.75) and incubated for 1 min at room temperature. Subsequently, 500 μl of 1 M aqueous guanidinium hydrochloride solution were added. Instantly, a precipitate started to form. Thereafter, the solution was centrifuged and the precipitate was further purified as described in Example 1.

### Results:

3 μg of nucleic acids (DNA and RNA) were isolated (OD_{260/280}=1.78).

All of the above patents, patent application, and non-patent publications referred to in this document are herewith incorporated by reference in their entirety.

1. A method to isolate or concentrate nucleic acids or nucleic acid analogs from an aqueous solution comprising the steps of:
(a) providing an aqueous solution containing nucleic acids,
(b) adding an aliquot of substance I to (a),
(c) adding an aliquot of substance II to (b), and
(d) centrifuge the aqueous solution of (c) and discard the supernatant,
wherein step (b) and step (c) are interchangeable or step (b) and step (c) can be performed at the same time, wherein an aliquot of substance I and an aliquot of substance II is given separated from each other but at the same time to the aqueous solution of (a).

2. A method according to claim 1, wherein substance I is chosen from the group of negatively charged ionic detergents.

3. A method according to claim 2, wherein substance I is lithium dodecyl sulfate (LiDS), sodium dodecyl sulfate (SDS) or a mixture thereof.

4. A method according to claim 1, wherein the final concentration of substance I is in a range of from 0.1% (w/v) to 10% (w/v).

5. A method according to claim 4, wherein the final concentration of substance I is in a range of from 0.4% (w/v) to 5% (w/v).

6. A method according to claim 5, wherein the final concentration of substance I is in a range of from 0.5% (w/v) to 1% (w/v).

7. A method according to claim 1, wherein substance II is a chaotropic salt or a mixture of different chaotropic salts.

8. A method according to claim 7, wherein substance II is selected from urea, sodium iodide, potassium iodide, sodium permanganate, potassium permanganate, sodium perchlorate, potassium perchlorate, sodium chlorate, potassium chlorate, guanidinium hydrochloride, guanidinium isothiocyanate, guanidinium thiocyanate, hexamine cobalt chloride, tetramethyl ammonium chloride, alkyltrimethyl ammonium chloride, tetraethyl ammonium chloride, tetramethyl ammonium iodide, alkyltrimethyl ammonium iodide, tetraethyl ammonium iodide or a mixture thereof.

9. A method according to claim 1, wherein the final concentration of substance II is in a range of from 0.1 M to 7 M.

10. A method according to claim 9, wherein the final concentration of substance II is in a range of from 0.2 M to 2 M.

11. A method according to claim 10, wherein the final concentration of substance II is in a range of from 0.25 M to 1 M.

12. A method according to claim 1, wherein substance I and substance II are chosen in that way, that they form a heterogeneous solution when brought together in one solution.

13. A method according to claim 1, wherein substance I and/or substance II are added as a solution.

14. A method according to claim 1; wherein substance I or substance II are added as solids.

15. A method according to claim 1, wherein the nucleic acid is DNA or RNA or a mixture thereof.

16. A method according to claim 1, wherein the precipitate obtained in step (d) of claim 1 is further purified.

17. A kit for performing a method according to claim 1 comprising:
(a) substance I, and
(b) substance II.

18. A kit according to claim 17 further comprising:
(c) a set of solutions or devices to further purify the nucleic acids contained in the precipitate obtained in step (d) of claim 1.
